(19) United States
(12) Patent Application Publication
(10) Pub. No.: US 2008/0119821 A1
(43) Pub. Date: May 22, 2008

(54) Multiport Cannula

(75) Inventors: Aashiish Agnihotri, Memphis, TN (US); Joseph Jude Saladino, Memphis, TN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/561,183

(22) Filed: Nov. 17, 2006

(51) Int. Cl. A61M 31/00; A61F 11/00; A61B 17/32

(52) U.S. Cl. 604/513; 604/510; 606/108; 606/167

(57) ABSTRACT

A cannula system for accessing a section of a vertebral column has a handle body and a tubular member extending from the handle body and adapted for percutaneous insertion into the vertebral column. The system has a first port in the handle portion in communication with a first passage which extends through the handle portion. The first passage is in communication with the interior bore. The system also has a second port in the handle portion in communication with a second passage which extends through the handle portion. The second passage is in communication with the interior bore.

### BACKGROUND

The present disclosure relates generally to systems and methods for accessing anatomic bodies and, more particularly, to systems and methods for delivering materials through cannulated access systems. Cannula systems for accessing anatomic structures to percutaneously carry instruments, evacuate tissue, or deliver materials are frequently used in medical procedures. Some cannula systems used for delivering fluid materials can be attached to and receive dispensed material from injection systems. Managing these multiple systems while maintaining proper positioning of the patient, providing physician access, and operating medical imaging equipment can be challenging. Improved material delivery and access systems are needed to accommodate these competing requirements.

### SUMMARY

In one embodiment, a system comprises a handle portion and a tubular member extending from the handle portion and adapted for percutaneous insertion into an anatomic structure. The tubular member defines a longitudinal axis and an interior bore. The system further comprises a first passage through the handle portion and in communication with the interior bore and a second passage through the handle portion and in communication with the interior bore. In certain embodiments multiple passages may extend through the handle portion in communication with the interior bore.

In another embodiment, a surgical method comprises the step of inserting a stylet through a first port in a cannula handle and into a cannula body and inserting at least a portion of the stylet and cannula body into a vertebral body. The surgical method further comprises withdrawing the stylet from the cannula handle and body and dispensing a material flow through a second port in the cannula handle and through the cannula body into the vertebral body.

In another embodiment, a system comprises a cannula body adapted to penetrate a vertebral body, the cannula body defining an interior bore extending along a longitudinal axis. The system further comprises a cannula handle connected to the cannula body. The cannula handle including a first port, a second port, a first passage extending through the cannula handle between the first port and the cannula body, and a second passage extending through the cannula handle between the second port and the first passage. The first passage extends along the longitudinal axis, in fluid communication with the interior bore.

Additional embodiments are included in the attached drawings and the description provided below.

### BRIEF DESCRIPTION OF THE DRAWINGS

FIG. 1 is a perspective view of a section of a vertebral column accessed by a cannula system.

FIG. 2 is a side view of a cannula system according to one embodiment of the present disclosure.

FIG. 3. is a side view of a multiple port cannula system according to another embodiment of the present disclosure.

FIG. 4 is a side view of a multiple port cannula system with a closed lateral port according to another embodiment of the present disclosure.

FIG. 5 is another side view of the multiple port cannula system, according to the embodiment of FIG. 4, with a closed longitudinal port.

FIG. 6 is a side view of a multiple port cannula system with an adjustable port according to another embodiment of the present disclosure.

FIG. 7 is another side view of the multiple port cannula system, according to the embodiment of FIG. 6.

FIG. 8 is a side view of a multiple port cannula system according to another embodiment of the present disclosure.

### DETAILED DESCRIPTION

For the purposes of promoting an understanding of the principles of the invention, reference will now be made to the embodiments, or examples, illustrated in the drawings, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

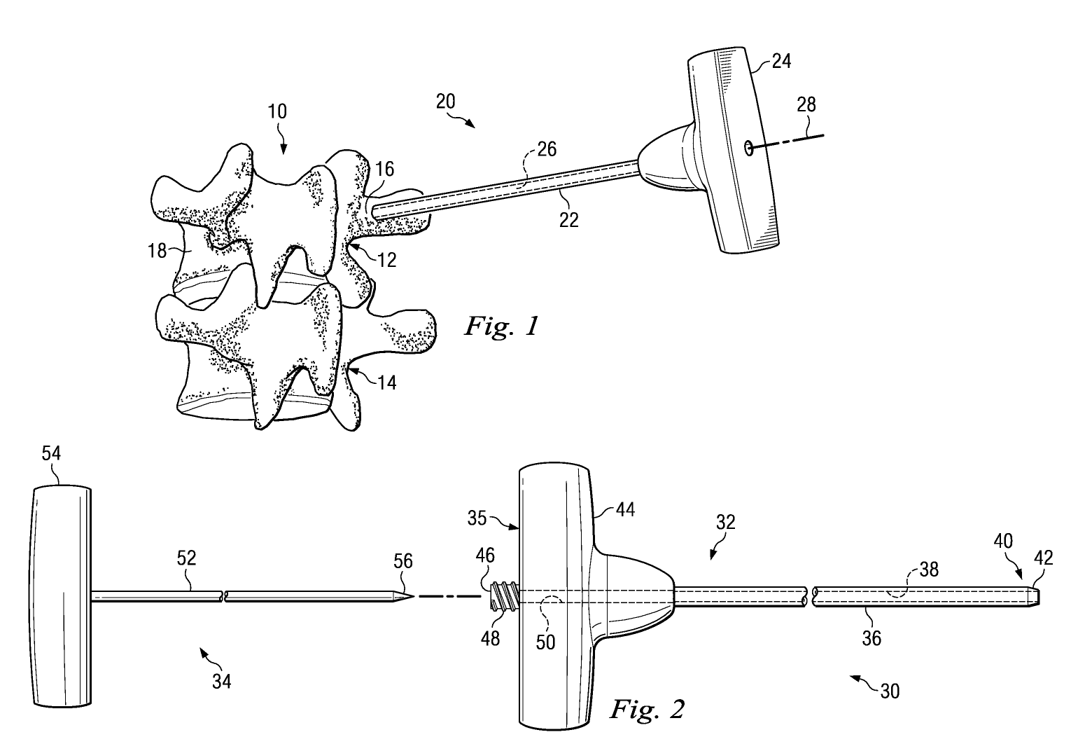

Referring first to FIG. 1, the reference numeral 10 refers to a segment of a vertebral column. The segment 10 includes adjacent vertebrae 12, 14. The vertebra 12 includes a pedicle 16 and a vertebral body 18. Although the instruments and methods described in this disclosure will generally reference a posterior surgical approach through the pedicle 16 and into the vertebral body 18, it is understood that, anterior, posterolateral, or anterolateral approaches may also be suitable. Furthermore, the instruments and methods described in this disclosure may administered to any region of the vertebral column including the lumbar or cervical region. It is to be understood, in a broad aspect, that the instrumentation and methods that will be described may be useful to treat a variety of anatomic structures, including other types of bones or soft tissue.

FIG. 1 additionally depicts a cannula assembly 20 comprising a cannula body 22 connected to a cannula handle 24. The cannula handle 24 may be rigidly or removably connected to the cannula body 22. The cannula body 22 may be generally tubular, defining an internal bore 26 which extends along a longitudinal axis 28.

Referring now to FIG. 2, in this embodiment, a percutaneous access system 30 includes a cannula assembly 32 which may receive a stylet assembly 34. The cannula assembly 32 may be similar to the cannula assembly 20 and may include a cannula handle 35 and a cannula body 36. In this embodiment, the cannula body 36 is tubular and defines an interior bore 38 extending the length of the cannula body. A distal portion 40 of the cannula body 36 may have a tapered end 42 to promote a smooth insertion into the vertebra 12. In this embodiment, the cannula body 36 may be rigid, however, in alternative embodiments, a cannula body may be flexible and/or compliant.

The cannula body 36 may be formed of a metal, polymer, ceramic, or composite material. Specifically, suitable materials may include metals such as cobalt-chromium alloys, titanium alloys, nickel titanium alloys, magnesium alloys, and/or stainless steel alloys. Suitable polymer materials may inlcude any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. Relatively rigid ceramic materials such as aluminum oxide or alumnia, zirconium oxide or zirconia, compact of particulate diamond, and/or pyrolytic carbon may be suitable.

The cannula handle 35 may include a gripping surface 44 shaped to enable a physician to firmly grasp the handle. The cannula handle 35 may further include a port or opening 46 which includes a connection feature 48 which in this embodiment is a luer lock connector having a set of outer threads for connecting the cannula handle to additional instruments. In certain embodiments, the connection feature may be a set of inner threads, a quick release locking mechanism, or other suitable connector. A passage 50 may extend through the cannula handle 35 between the port 46 and the cannula body 36. The passage 50 may communicate with the interior bore 38, permitting passage of instruments or a material flow from the port 46 into the cannula body 36. In this embodiment, the passage 50 is coaxial with the interior bore 38, but in alternative embodiments, a passage through the cannula handle 35 may be angled relative to the interior bore of the cannula body.

The cannula handle 35 may be formed of any suitable material including metals, polymers, ceramics, or composite materials. For example, the cannula handle may be formed of a molded plastic. The passage 50 may be defined by the material of the cannula handle 35 or may, alternatively, be defined by a tube formed of a different material, such as a metal or polymer tube, extending through the cannula handle. In the embodiment of FIG. 2, the cannula body 36 may be rigidly connected affixed to the cannula handle 35, such as by overmolding, but in certain embodiments, the cannula body may be removable.

The stylet assembly 34 may include an elongated shaft 52 extending between a stylet handle 54 and a distal tip 56. The distal tip 56 may be conical, beveled, or of another sharpened shape suitable for accessing the desired anatomic structure. The stylet may be formed of any of any suitable material including the rigid materials listed above.

The percutaneous access system 30 may be assembled by inserting the distal tip 56 of the stylet assembly 34 into the port 46 of the cannula handle 35. The shaft 52 may extend through the passage 50 and the interior bore 38 with the distal tip 56 protruding from the tapered end 42 of the cannula body 36. The stylet shaft 52 and the cannula body 36 may together be described as a surgical needle. Small size needles such as, for example, 13 gauge or smaller with outside diameters of approximately 2.4mm may be suitable. Larger or smaller needles may also be appropriate. The stylet handle 54 may include inner threads (not shown) which mate with the connection feature 48 to secure the stylet assembly 34 to the cannula assembly 32.

In use, the assembled percutaneous access system 30 may be used to access a vertebral body and locate the cannula assembly 32 for later use in delivering instruments or flowable materials to the vertebral body 18. Specifically, with the aid of diagnostic imaging equipment such as fluoroscopy, a physician may locate the pedicle 16 of the vertebra 12 and the related vertebral body 18 (See FIG. 1). Then, in a minimally invasive manner, the distal tip 56 of the stylet assembly 34 may be used to penetrate the skin of the patient and further advance the combined cannula body 36 and shaft 52 through the skin, into the pedicle 16, and into the vertebral body 18. The stylet assembly 34, in addition to providing the sharp point needed to penetrate tissue, also provides rigidity to the cannula body 36, allowing the combined instrument to pass through hard tissue without significant deformation. After the cannula body 36 is positioned in the vertebral body 18, the stylet assembly 34 may be withdrawn, leaving the cannula body extending out of the patient.

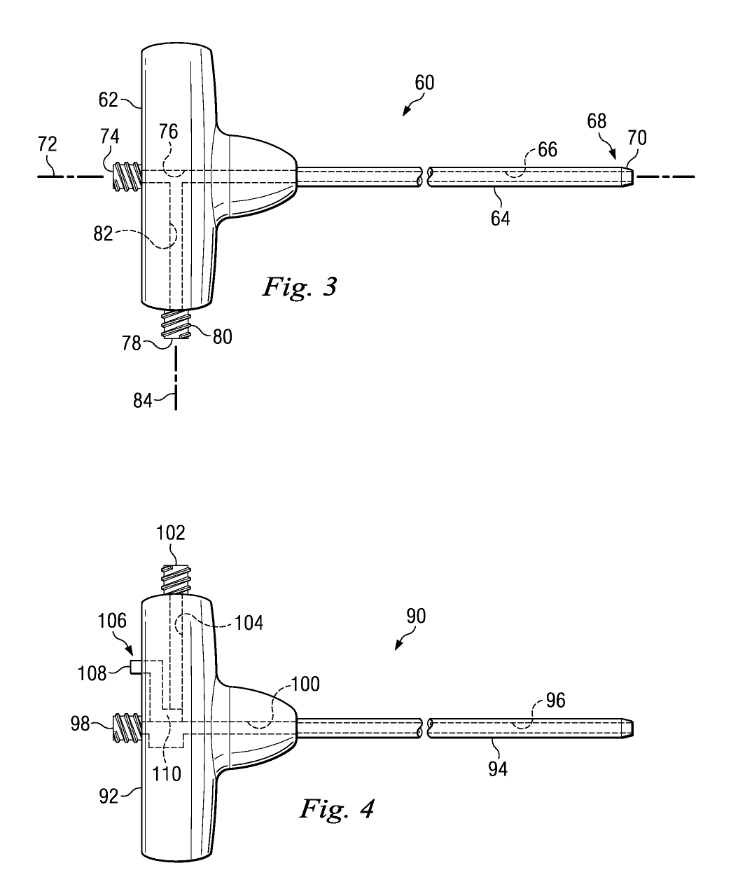

Referring now to FIG. 3, a cannula assembly 60 may be substantially similar to the cannula assembly 32 with the differences to be noted. The cannula assembly 60 may include a cannula handle 62 and a cannula body 64. In this embodiment, the cannula body 64 is tubular and defines an interior bore 66 extending the length of the cannula body. A distal portion 68 of the cannula body 64 may have a tapered end 70 to promote a smooth insertion into the vertebra 12. As illustrated, the elongated cannula body 64 extends lengthwise along a longitudinal axis 72.

The cannula handle 62 may include a port 74. A passage 76 may extend through the cannula handle 62 between the port 74 and the cannula body 64. The passage 76 may communicate with the interior bore 66, permitting passage of instruments or a material flow from the port 74 into the cannula body 64. The passage 76 and the port 74 may be coaxial with longitudinal axis 72.

In this embodiment, the cannula handle 62 may further include a lateral port 78 which includes a connection feature 80 which in this embodiment is a luer lock connection having a set of outer threads for connecting the cannula handle to additional instruments. In certain embodiments, the connection feature may be a set of inner threads, a quick release locking mechanism, or other suitable connector. A passage 82 may extend through the cannula handle 62 between the port 78 and the passage 76 such that the passage 82 is in communication with the interior bore 66, permitting passage of instruments or a material flow from the port 78 into the cannula body 64. In this embodiment, the passage 82 extends along an axis 84 which orthogonally intersects the longitudinal axis 72. Thus, the ports 74, 78 are disposed at approximately right angles to one another. In alternative embodiments, the passages may define axes that intersect at oblique angles. Although only two passages through the cannula handle 62 will be described, it is understood that additional passages may extend through the cannula handle such that multiple ports in the cannula handle may be used to access the interior of the cannula body 64.

In use, the cannula assembly 60 may be deployed inside a vertebral body 18 as described above for cannula assembly 32. As deployed, the longitudinal axis 72 may be generally coincident with the longitudinal axis 28 (see FIG. 1). With the cannula assembly 60 inserted in the vertebral body 18, the interior bore 66 may function as a conduit for the passage of materials or instruments into the vertebral body.

One example of a procedure that may be performed using the deployed cannula assembly 60 is vertebroplasty, a treatment in which bone cement or other material capable of setting to a hardened condition is delivered into the vertebral body to support osteoporotic bone, restore height to a vertebral compression fracture, fill bone cavities created by tumors, or otherwise correct a malady of the vertebral body. Although the embodiments of this disclosure will generally describe vertebroplasty procedures, in alternative embodiments, the cannula assembly may be used to deliver instruments and materials for other procedures such as kyphoplasty or bone void creation.

With the cannula body 64 deployed in the vertebral body 18, a material injection system may be connected to the port 78 by the connection feature 80 to dispense flowable material such as bone cement into the passage 82. Any of a variety of material injection systems may be used including components of the Medtronic ARCUATE™ System (distributed by Medtronic,Inc. of Minneapolis, Minn.) or those described in U.S. Pat. Nos. 6,383,190 and 6,348,055 which are incorporated by reference herein. The material injection system may be connected via a rigid connection such that, once connected, little or no movement occurs between the connection feature and the material injection system. In certain embodiments, movement between the connection and feature and the material injection system may be supplied by, for example, a rotating luer connection on the material injection system, or a flexible tube connecting the two instruments. Suitable bone cement materials may include polymethylmethacrylate (PMMA) or calcium phosphate compositions.

The flowable material, which may be dispensed under pressure, may flow through the passage 82, into the passage 76, into the interior bore 66 and out through the tapered end 70 into the vertebral body. The port 74 may be capped or plugged to prevent the flowable material from dispensing out the port 74. In certain embodiments, a flowable material may be dispensed through the port 74 in addition to or as an alternative to port 78. Use of the laterally oriented port 78 may be particularly useful in procedures in which the physician wishes to avoid shadows on the fluoroscopic images that might be generated by a material injection instrument located near or in line with the longitudinal axis 72. Furthermore, because the patient is frequently positioned in a prone position with the cannula system extending nearly vertically from the patient's back, and the physician located near the patient's side, direct access to the longitudinal port 74 may be challenging. Flexible tubing, bent tubing, or other extension devices may be attached to the longitudinal port to allow the physician to comfortably access the port 74. These extension devices must be carefully selected to withstand the high pressures generated by the material injection instrument. Even when selected to avoid the risk of rupture, extension devices add resistance which can make stopping or starting the material flow more difficult. Furthermore, extension devices may make delivering exact quantities of bone cement difficult because material may be retained in the extension device. Use of the lateral port 78, which may be closer to the physician, may reduce or eliminate the need for tubing or other extension devices that can reduce control over the initiation or stopping of the material flow. Connecting a material delivery system directly to the lateral port 78 or with only limited amounts of extension tubing may provide better control over the amount and the location of the material deposited.

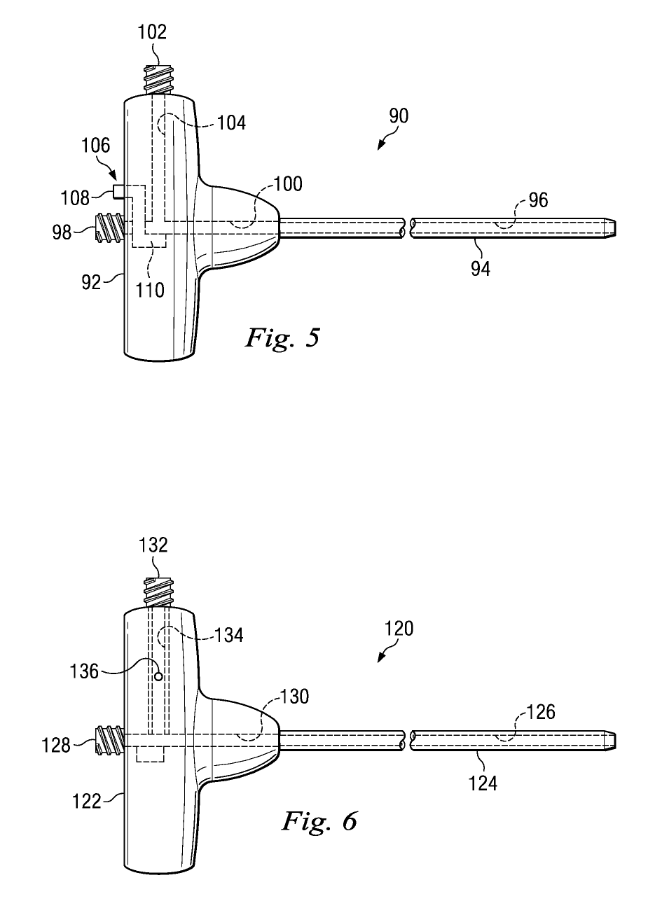

Referring now to FIGS. 4 and 5, a cannula assembly 90 may be substantially similar to the cannula assembly 60 with the differences to be noted. The cannula assembly 90 may include a cannula handle 92 and a cannula body 94. In this embodiment, the cannula body 94 is tubular and defines an interior bore 96 extending the length of the cannula body.

The cannula handle 92 may include a port 98. A passage 100 may extend through the cannula handle 92 between the port 98 and the cannula body 94. The passage 100 may communicate with the interior bore 96, permitting passage of instruments or a material flow from the port 98 into the cannula body 94. The passage 96 and the port 98 may be coaxial.

In this embodiment, the cannula handle 92 may further include a lateral port 102. A passage 104 may extend through the cannula handle 92 between the port 102 and the passage 100 such that the passage 104 is in communication with the interior bore 96, permitting passage of instruments or a material flow from the port 102 into the cannula body 94. In this embodiment, the passage 104 orthogonally intersects the passage 100.

The cannula handle 92 may further include a valve assembly 106 which may include a valve actuator 108, such as a knob, extending from the cannula handle 92 and a blocking arm 110 connected to or otherwise responsive to activation by the valve actuator. As shown in FIG. 4, in one position, the blocking arm 110 may obstruct the passage 104 while the passage 100 remains unobstructed. In this position, materials or instruments may pass through the passage 100 into the interior bore 96 but may be partially or completely prevented from passing through passage 104 into the passage 100. As shown in FIG. 5, the valve actuator 108 may be selectively activated using a manual, electric, pneumatic or another power source to move the blocking arm 110 into another position such that the blocking arm obstructs a portion of the passage 100 between the passage 104 and the port 98. In this position, materials or instruments may pass through the passage 104 into the passage 100 and into the interior bore 96 but may be partially or completely prevented from passing through the passage 100 between the passage 104 and the port 98. The blocking arm 110 may slide, rotate, swing, or otherwise move between blocking positions when activated by the valve actuator 108. Gaskets or other sealing devices (not shown) may be used to prevent material flow around the blocking arm or the valve actuator. Although only two passages through the cannula handle 92 are described, it is understood that additional passages and corresponding valve assemblies may be included in the cannula handle such that multiple ports in the cannula handle may be used to access the cannula body.

In use, the cannula assembly 90 may be deployed into the vertebral body 18 as described above for cannula assembly 60. With a stylet assembly inserted into the cannula assembly 90, the blocking arm may be positioned to leave the passage 100 unobstructed. When the stylet assembly is removed, flowable material may be dispensed through either port 98 or 102. If material is dispensed through the port 98, the blocking arm 110 can remain positioned as shown in FIG. 4 such that the passage 104 is blocked. If material is dispensed through the port 104, the valve actuator 108 may be activated to move the blocking arm 110 into a position, as shown in FIG. 5, in which the passage 104 is unobstructed, allowing the flow of material therethrough and into the passage 100.

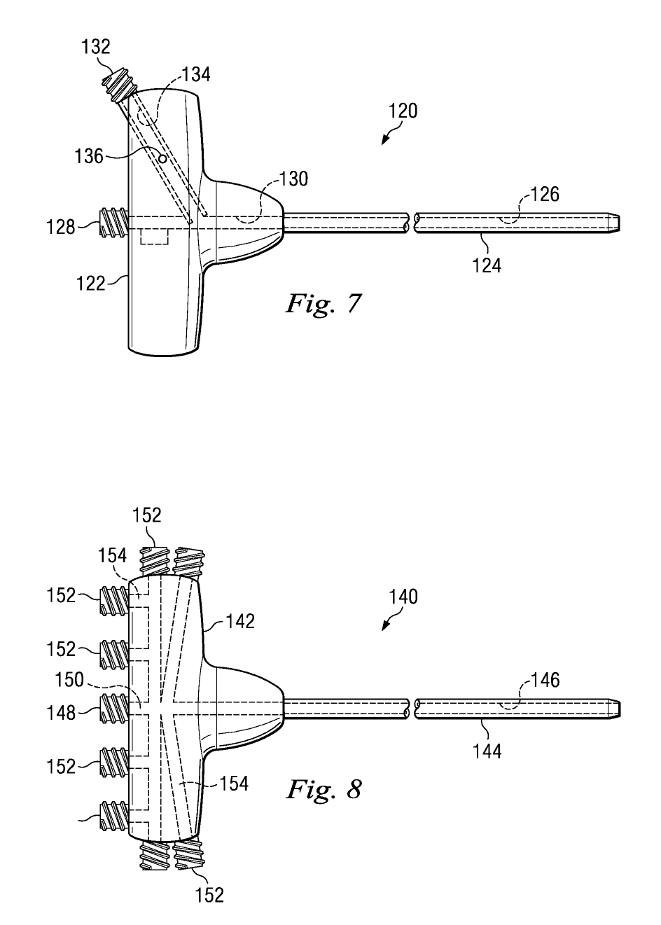

Referring now to FIGS. 6 and 7, a cannula assembly 120 may be substantially similar to the cannula assembly 60 with the differences to be noted. The cannula assembly 120 may include a cannula handle 122 and a cannula body 124. In this embodiment, the cannula body 124 is tubular and defines an interior bore 126 extending the length of the cannula body.

The cannula handle 122 may include a port 128. A passage 130 may extend through the cannula handle 122 between the port 128 and the cannula body 124. The passage 130 may communicate with the interior bore 126, permitting passage of instruments or a material flow from the port 128 into the cannula body 124. The passage 126 and the port 128 may be coaxial.

In this embodiment, the cannula handle 122 may further include a port 132. A passage 134 may extend through the cannula handle 134 between the port 132 and the passage 130 such that the passage 134 is in communication with the interior bore 126, permitting passage of instruments or a material flow from the port 132 into the cannula body 124.

The cannula handle 122 may further include a movement mechanism 136 which can be activated to move the port 132 relative to the port 128. As shown in FIG. 6, in a first position, the passage 134 may be generally orthogonally disposed relative to the passage 130. As shown in FIG. 7, the movement mechanism 136, which in this embodiment may be a pivot mechanism, may be activated to rotate the port 132 and the passage 134 relative to the port 128 and the passage 130, respectively, such that the passage 134 is disposed at an oblique angle relative to the passage 130. Other movement mechanisms such as sliding mechanisms may be suitable to adjust the lateral port relative to the longitudinal port. In this embodiment, the pivot mechanism may permit rotational movement of the passage 134 in a single plane, however, in alternative embodiments, the passage and the movement mechanism may permit translational motion in the plane of rotation. In still other alternative embodiments, the movement mechanism may permit movement in multiple planes.

In use, the cannula assembly 130 may be deployed into the vertebral body 18 as described above for cannula assembly 60. A material injection system, as described above, may be attached to the port 132, and the movement mechanism 136 may be activated to position the material injection system in a location that is both comfortable for physician use without interfering with imaging equipment. In certain embodiments, the movement mechanism may be locked in place once the desired angle of the passage 134 is achieved, but in other embodiments, the movement mechanism may remain free to move to accommodate the movement of the physician. Suitable locking mechanisms may include a depressable button or a slidable knob to prevent rotation of the passage 134. In certain alternative embodiments, the passage 130 and/ or the passage 134 may be lined with flexible tubing that flexes as the passage 134 is moved relative to the passage 130. In certain embodiments, for example as shown in FIG. 7, the movement of the passage 134 may completely or partially obstruct the passage 130 to prevent material from escaping through port 128. Although only two passages through the cannula handle 122 are described, it is understood that additional movable passages may extend through the cannula handle such that multiple ports in the cannula handle may be used to access the cannula body.

Referring now to FIG. 8, a cannula assembly 140 may be substantially similar to the cannula assembly 60 with the differences to be noted. The cannula assembly 140 may include a cannula handle 142 and a cannula body 144. In this embodiment, the cannula body 144 is tubular and defines an interior bore 146 extending the length of the cannula body.

The cannula handle 62 may include a port 148. A passage 150 may extend through the cannula handle 142 between the port 148 and the cannula body 144. The passage 150 may communicate with the interior bore 146, permitting passage of instruments or a material flow from the port 148 into the cannula body 144. The passage 150 and the port 148 may be coaxial with the longitudinal axis of the cannula body 144.

In this embodiment, the cannula handle 142 may further include a plurality of ports 152 which include connection features such as those described above. A plurality of passages 154 may extend through the cannula handle 142 between the ports 152 and the passage 150 such that the passages 154 are in communication with the interior bore 146, permitting passage of instruments or a material flow from the ports 152 into the cannula body 144. It is understood that any of the passages 150, 154 or ports 148, 152 may be blocked using valve assemblies such as those described above or may be movable using any of the movement mechanisms described above. The cannula assembly 140 may be deployed inside the vertebral body 18 and used to dispense material as described above. It is understood that the number of ports and passages described are exemplary and fewer or more ports and passages may be included in the cannula assembly as may be suitable to the procedure to be performed.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top ,” “upper,” “lower,” “bottom,” “left,” “right,” “anterior,” “posterior,” “superior,” “inferior,” “upper,” and “lower” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A surgical instrument system comprising:
a handle portion;
a tubular member extending from the handle portion and adapted for percutaneous insertion into an anatomic structure, the tubular member extending along a longitudinal axis and including an interior bore;
a first port in the handle portion in communication with a first passage extending through the handle portion, wherein the first passage is in communication with the interior bore; and
a second port in the handle portion in communication with a second passage extending through the handle portion, wherein the second passage is in communication with the interior bore.

2. The system of claim 1 wherein the second passage is in communication with the first passage.

3. The system of claim 1 wherein the first passage is in direct communication with the interior bore.

4. The system of claim 1 wherein first passage includes a proximal end in direct communication with interior bore and includes a distal end in direct communication with the first port and further wherein the second passage intersects the first passage between the proximal end and the distal ends.

5. The system of claim 1 wherein the first port is disposed about the longitudinal axis and the second port is disposed along a second axis that intersects the longitudinal axis.

6. The system of claim 5 wherein the second axis is perpendicular to the longitudinal axis.

7. The system of claim 1 further comprising a valve configured to selectively block communication between at least a section of the first passage and the interior bore while maintaining communication between the second passage and the interior bore.

8. The system of claim 7 wherein the valve is further configured to move to selectively block communication between at least a section of the second passage and the interior bore while maintaining communication between the first passage and the interior bore.

9. The system of claim 7 wherein the valve is configured to slide between a first position and a second position.

10. The system of claim 7 wherein the valve is configured to pivot between a first position and a second position.

11. The system of claim 1 wherein the second passage is movable with respect to the first passage.

12. The system of claim 11 further comprising a pivot mechanism wherein the second passage is movable with respect to the first passage about the pivot mechanism.

13. The system of claim 1 wherein the anatomic structure is a bone.

14. The system of claim 1 wherein the anatomic structure is a vertebral body.

15. The system of claim 1 further comprising a stylet adapted for insertion through the first passage and the interior bore.

16. The system of claim 1 further comprising a third port in the handle portion in communication with a third passage extending through the handle portion, wherein the third passage is in communication with the interior bore.

17. A surgical method comprising:
inserting a stylet through a first port in a cannula handle and into a cannula body;
inserting at least a portion of the stylet and cannula body into a vertebral body;
withdrawing the stylet from the cannula handle and body; and
dispensing a material flow through a second port in the cannula handle and through the cannula body into the vertebral body.

18. The surgical method of clam 17 further comprising:
attaching a material dispensing device to the second port in the cannula handle.

19. The surgical method of claim 18 further comprising providing a non-flexible connection between the material dispensing and the cannula handle.

20. The surgical method of claim 17 further comprising:
moving the second port relative to the first port.

21. The surgical method of claim 17 further comprising:
operating a valve to obstruct the first port during the dispensing of the material flow through the second port.

22. A surgical access system comprising:
a cannula body adapted to penetrate a vertebral body, the cannula body defining an interior bore extending along a longitudinal axis and
a cannula handle connected to the cannula body, the cannula handle including a first port, a second port, a first passage extending through the cannula handle between the first port and the cannula body, and a second passage extending through the cannula handle between the second port and the first passage;
wherein the first passage extends along the longitudinal axis, in fluid communication with the interior bore.

23. The system of claim 22 further comprising:
a valve assembly comprising a blocking arm configured to move between a first position in which the blocking arm obstructs the first passage and a second position in which the blocking arm obstructs the second passage.

24. The system of claim 22 further comprising:
a movement mechanism configured to move the first port relative to the second port.

25. The system of claim 24 wherein the movement mechanism is a pivot mechanism.

26. The system of claim 22 wherein the cannula handle is rigidly connected to the cannula body.

27. The system of claim 22 further comprising:
a valve assembly operative to restrict flow through a selectively variable one of the first and second passages.
